(19) United States
(12) Patent Application Publication
(10) Pub. No.: US 2009/0060799 A1
(43) Pub. Date: Mar. 5, 2009

(54) Adjustable tabletop air freshener

(76) Inventor: David Fernandez Torres, Huercal De Almerla (ES)

Correspondence Address: JACOBSON HOLMAN PLLC, 400 SEVENTH STREET N.W., SUITE 600, WASHINGTON, DC, 20004, US

(21) Appl. No.: 11/785,053

(22) Filed: Apr. 13, 2007

(30) Foreign Application Priority Data
Aug. 9, 2006 (ES) U200601877

(51) Int. Cl. A61L 9/12

(52) U.S. Cl. 422/123

(57) ABSTRACT

We describe an adjustable table-top air freshener composed of a case that comprises an internally hollow body, provided with four pairs of slots arranged in couples around the perimeter of its lateral wall, and a cover which presents through-holes in its surface. The case body is designed to hold four absorbent pieces of aromatizing product, executed in twos with a three-point star and circular configuration with alternately sunken and protruding portions on its perimeter edge, of a diameter slightly smaller than the inside diameter of the case body and stacked alternately in the interior of the case body, with a star-shaped piece occupying the bottom position. The cover presents the formation of ribs of a predetermined height protruding from its inner face, interposed respectively between successive openings, which assure the separation between said cover and the absorbent piece located in the upper position.

### OBJECT OF THE INVENTION

The present invention refers to a new adjustable air freshener consisting of an openable box of any configuration, forming a table-top air freshener, where the actual box comprises two parts that may be fitted together, one of them the actual box and the other the top cap housing in its interior pieces of cellulose material impregnated with perfume.

### BACKGROUND OF THE INVENTION

The prior art comprises the existence of different types of air freshener in hanging, pedestal and tabletop form, both constant evaporation and adjustable type, but all the means currently used, their service life and the quality of their air freshening effect are extremely limited, as for different reasons the diffusion of the perfume proves highly limited in view of the occlusion presented by both their internal and external components, whilst the adjustable tabletop air freshener that is the object of the present registration eliminates all the defects found, facilitating adjustment of the discharge of the perfume as the internal laminar absorbent pieces present balanced perimetral conformations that assist the regulated evaporation of the perfume, which, for this purpose, the box that contains the air freshener product acts by convection, with a lower air intake by way of peripheral slots, issuing from the top of the cap by way of helical holes made therefor.

### DESCRIPTION OF THE INVENTION

The adjustable tabletop air freshener that is the object of the present invention is made up of a circular box, although it could also take on any other shape, such as square, oval, rectangular, etc., said circular box consisting of two parts, the base or body of the box and the cap.

The body or base of the box has four pairs of ventilation slots in the lower half of the circular peripheral wall and all along its perimeter, so that each pair of ventilation slots is separated by a kind of strip of slender material, the aforesaid slots presenting a rectangular design although they could also have other designs of a circular, oval or any other shape.

A first piece with a perimetral three-armed star shape of an absorbent spongy material approximately 5.8 millimetres thick is placed initially inside the base of the box through its mouth. This thickness may vary, the maximum diameter of this star-shaped piece being slightly smaller than the inside diameter of the box, so that this star-shaped piece is always positioned concentrically inside the circular box. A second piece is then set n place, also made of the same absorbent spongy material of the same thickness but of a different design, as it presents a circular configuration with four projections that fit the inside diameter of the box with a suitable tolerance, while incorporating alternately four recesses, following the perimeter of a concentric inner circle.

A third piece, of the same star shape as the first piece fitted is then inserted and afterwards a fourth and last piece is introduced of a circular design the same as the piece fitted in second place, so that the set of absorbent pieces in the box is ready to receive the perfume, duly dispensed in the proper amount, which is partly absorbed by the top piece, while the rest descends down the four recesses in the circular absorbent pieces, impregnating the whole of the absorbent pieces, as the liquid perfume drops to the bottom of the circular receptacle and is absorbed by the bottom piece, which transmits it in turn in an ascending direction by capillarity to the different absorbent pieces arranged in a superimposed fashion, the action of this kind of impregnation being such that the liquid perfume descends first by gravity, gradually impregnating the four pieces, and then the perfume ascends by capillarity and is spread among the four pieces, ensuring that they are all impregnated with perfume.

In this adjustable air freshener there are two types of absorbent pieces and the design of each one of these pieces fulfils specific functions as the three-point star-shaped pieces have this design in order that they may have the least possible surface area while at the same time their three arms prevent the piece from moving inside the box. The purpose of the star-shaped piece positioned at the bottom of the box is to separate the circular piece with projections and recesses disposed in superimposed fashion on the base of the box in order that the perfume may evaporate on its under surface, while another function of the star-shaped piece of the box base is to absorb the liquid perfume from the initial dispensing of the whole system and that part which has remained at the bottom of the box, transmitting it by capillarity to the upper piece.

The pieces in the form of a circle with four symmetrical projections and recesses are designed in this way so that they have the largest surface area possible, while their four projections prevent the piece from moving inside the box and their four recesses allow the liquid perfume to drop down through them when the initial dispensing takes place on the fourth cellulose plate. These four recesses are the ones that also allow the evaporated perfume from the lower cellulose layers to pass by them in an upward direction. The function of the intermediate star-shaped triangular piece is to separate the upper and lower circular absorbent pieces, so that they may evaporate on their upper and lower surfaces, respectively. During the process of dispensing the liquid perfume to the whole system, this intermediate star-shaped piece in contact with the upper and lower circular pieces acts as a transmitter of liquid perfume between both by capillarity.

Once all the laminar absorbent cellulose pieces are in place in the box and the measure of liquid perfume has been applied, the cap is then press fitted on the box. In its central inner part this cap has six ribs projecting vertically in relation to the plane of the cover and situated in each of the six spaces between the six blade-like windows that are provided in the cap, the function of these ribs being to exert pressure on the upper cellulose piece in respect of the other superimposed pieces, maintaining a parallel separation between the upper surface of the cellulose piece and the inner plane of the cap, so that there is a flow of air and perfume in this gap between both surfaces, whilst also allowing the diffusion of the perfume that ascends up the space next to the circular inner wall of the body of the box to issue forth.

### DESCRIPTION OF THE FIGURES

For a clearer understanding of the general features described above, drawings are attached that show us, represented graphically and diagrammatically, a case of a practical preferred embodiment of the adjustable tabletop air freshener that is the object of the invention, while pointing out that, in view of the eminently informative nature of the drawings in question, the figures designed in them should be examined with an extremely comprehensive attitude and on a no way restrictive basis. In these figures there is represented the following:

FIG. 1 shows a perspective projection of the air freshener with all the absorbent pieces making up its composition designed correctly on a vertical axis, consisting of the lower box where the absorbent cellulose pieces will be housed in the following ascending order: first a three-pointed star-shaped piece is inserted, then a circular piece provided alternately on its perimeter edge with four recesses and four projections, incorporating next a second star-shaped piece and finally in the upper part of the group of cellulose pieces, another circular piece like the preceding one. The perfume is then added to the air freshener in liquid form and the unit is completed with a press-fit top cap.

FIG. 2 shows a perspective projection of the air freshener sealing cover seen exteriorly, presenting a plurality of through-holes that take on the form of a fan blade for the discharge of the air freshener by evaporation.

FIG. 3 shows a perspective view of the cap seen from its internal plane, showing the arrangement of the six projecting ribs that press on the upper cellulose plate, while the actual cap incorporates in its lower portion in the inner ring a female groove all round its perimeter which serves for engaging the O-ring disposed exteriorly on the top part of the body of the box.

FIG. 4 shows a perspective view of the adjustable tabletop air freshener provided with a self-adhesive of special plastic material on the top part of the cap, covering the six blade-like windows and another adhesive of the same plastic material around the circular wall of the body of the box at the bottom end, covering up all the slots to prevent the perfume from evaporating when the air freshener is not in service.

FIG. 5 shows a perspective view of the air freshener according to FIG. 4 without the upper self-adhesive label, maintaining the lower self-adhesive label covering the peripheral air intake slots partly stuck in place, with a normal diffusion of the perfume.

FIG. 6 shows a perspective view of the air freshener with all the absorbent self-adhesive pieces removed, in which case we obtain an evaporation of the perfume contained that may be termed as “intense”.

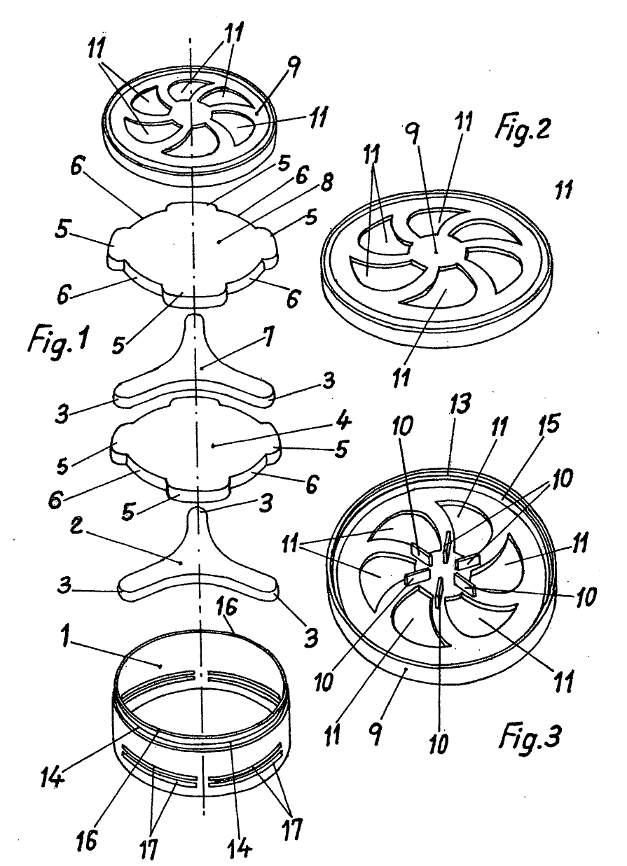

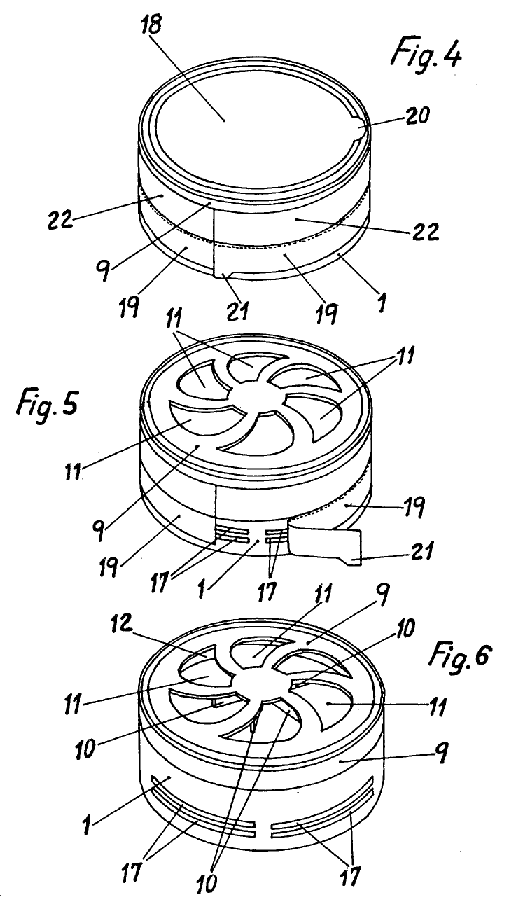

### PREFERRED EMBODIMENT OF THE INVENTION

In reference always to the adjoining drawings, it should be pointed out that in the different figures designed in them numerical elevational values are included relating to the descriptions of their features and operation given below, thereby facilitating their immediate location, (1) being the box of circular configuration or of any other geometrical figure, in which various laminar pieces of cellulose material are housed. The piece in the form of a three-armed star (2) of a spongy absorbent material with an approximate thickness of 5,8 millimetres is inserted first, the edges of the points of the star being of rounded configuration (3) and with a maximum diameter slightly less than the inside diameter of the box (1), so that the star is always positioned concentrically to the interior of the box.

The second piece is then inserted, likewise of the same spongy absorbent material (4) of the same thickness but of different design from piece (2), the design of the piece (4) being of circular form with four projections (5) matching the inside diameter of the interior of the box (1), alternating with four recesses (6), following the perimeter of an inner concentric circle.

A third star-shaped piece (7) is then fitted, identical to the piece inserted first (2), and lastly a fourth piece (8) of circular design, identical to the piece (4) installed in second place, is installed.

Once all the laminar absorbent material pieces (2), (4), (7) and (8) are in place in the box (1), the perfume is dispensed over the piece (8), so that the perfume applied in appropriate doses is absorbed partly by the upper piece (8) and the rest drops past the recesses (6) in the circular piece (8), impregnating the absorbent pieces (7), (4) and (2). The liquid perfume drops to the bottom of the circular receptacle (1) by way of the recesses (6) in the circular pieces (8), being absorbed by the piece (2), and this in turn also transmits the perfume to piece (4) in an ascending direction by capillarity, so that the perfume impregnates piece (7) too, and this in turn transmits it to the upper piece (8), i.e. first the liquid perfume descends by gravity and goes on impregnating the four cellulose pieces and then the perfume ascends by capillarity and is spread over the four pieces, ensuring that they are all impregnated with perfume.

There are two types of absorbent cellulose pieces and the design of each of these pieces fulfils specific functions, so that the pieces in the form of a three-pointed star (2) and (7) have this design in order that they may have the smallest surface area possible, while their three arms prevent movement in the box (1), the purpose of the piece (2), which has the aforesaid triangular shape, being that of separating the circular-shaped piece (4) from the bottom of the box (1) so that the perfume may evaporate on the under surface of said piece (4), while another purpose of piece (2) is that of absorbing the liquid perfume from the initial dispensing of the whole system as well as that perfume which has remained at the bottom of the box (1), transmitting it by capillarity to piece (4).

The circular-shaped absorbent pieces (4) and (8) with four symmetrical projections (5) and four recesses (6) have this design so that they may have the largest surface area possible, while their four projections (5) prevent movement within the box (1) and their four recesses (6) allow the liquid perfume to descend past them when the initial dispensing takes place on the fourth cellulose plate, these four recesses (6) being the ones that also enable the evaporated perfume from the lower cellulose layers to pass by them in an upward direction. The function of the cellulose piece (7) of the same triangular star shape as piece (2) is that of separating the circular absorbent pieces (4) and (8) so that they may evaporate on their upper and lower surfaces, respectively; during the process of dispensing liquid perfume to the whole system, this piece (7) in contact with the absorbent pieces (4) and (8) acts as a transmitter of liquid perfume between both by capillarity.

Once the absorbent pieces (2), (4), (7) and (8) are in place in the body of the box (1) and the perfume dispensed, the cap (9) is then press-fitted on the body of the box (1). The cap (9) is provided with six ribs (10) projecting from the lower plane of the actual cap, disposed vertically in relation to the plane of the cap and situated in each of the six spaces between the six windows (11) in the form of fan blades made in the upper surface of the cap, the function of these ribs being to exert pressure on piece (8) in respect of the other absorbent pieces (7), (4) and (2), holding piece (8) in a position parallel to the cap. Another function of these ribs (10) is to keep the upper surface of piece (8) separated from the lower surface of the cap (9), so that there is a circulation of air and perfume in this gap (12) between both surfaces, thereby also permitting the discharge of the diffusion of the perfume ascending up the space adjoining the inner circular wall of the body of the box, while in the inner ring at the bottom the actual cap (9) is also provided with a female groove (13) all round its outside edge that is used for engaging the O-ring (14) in the outer perimeter of the mouth of the body of the box (1), in the same way as the cap (9) is likewise provided in its lower portion with a concentric peripheral overlap (15) lower than the outer sealing overlap and separated from it by a small space, it being in this space where the circular rim (16) of the body of the box (1) is inserted at the time when the box is closed with the pressure cap (9).

In its vertical wall, in the form of an arc at a point close to the bottom of the box, the body of the box (1) has eight rectangular slots (17), arranged in four superimposed pairs spread appropriately all round the perimeter of the box, although these openings may vary in shapes and sizes that fulfil the same function, as the function of the actual slots is crucial for the system of air flow and diffusion of the perfume, constituting one of the most important elements of the air freshener, since the air enters via these slots (17) to the interior of the box (1) at the height of the cellulose piece (2) and rises gradually to the levels of the absorbent pieces (4), (7) and (8).

The air that enters via the slots (17), acts on all the free surfaces, i.e. not covered by one another, formed of the four absorbent pieces, producing the evaporation and diffusion of the perfume, which rises and issues from the six blade-like windows (11) in the cap (9). This air passes through the system of four levels of superimposed absorbent plates that act as a “radiator”, facilitating the evaporation of the perfume on its surfaces and thereby assisting its diffusion to the exterior of the box.

So that the air freshener does not evaporate perfume until the time when the decision is made to set it into operation, a self-adhesive label (18) of a special plastic material is fitted on the upper part of the cap, covering up the six blade-form windows (11) and another self-adhesive label (19) of the same material disposed around the circular wall of the body of the box (1), applied on the lower portion to cover up all the slots (17), both self-adhesive labels (18) and (19) having a tab (20) and (21), respectively, that is used for pulling on and thereby detaching the labels more easily.

This system of two self-adhesive labels for covering up the upper windows (11) with one of them (18) and the lateral slots (17) with the other (19) will enable us to control the degree of evaporation of the system. Thus, if only the upper label (18) is detached keeping the lateral one stuck in place, we obtain normal diffusion of the perfume, whereas if both labels are detached, the evaporation of perfume will be intense.

When sealed with the cap (9), the box (1) may have a third label (22) fitted, taking in the whole of the lateral perimeter of the cap (9) and the upper portion of the outer wall of the box (1), for the purpose of stopping the box cap form being opened and for the printing on the outer side of information on how to use the system.

Considering each and every one of the parts making up the hanging box with a built-in air freshener that is the object of the invention more than adequately described, it only remains for us to point out that the possibility that its different parts may be manufactured in a variety of materials, sizes, shapes and colours, while those variations of a constructional type may be introduced into its execution that practice so advocates, providing that they are not capable of altering the essential points of what is the object of the present Utility Model registration.

1. An adjustable tabletop air freshener, characterised in that it comprises a box, either circular or of any other geometrical shape, which has pairs of superimposed ventilation slots, separated by a narrow strip of material of the actual box, made in its lower portion and arranged equidistantly all round its periphery.

2. The adjustable tabletop air freshener, according to the previous claim, characterised in that it comprises plates of porous material of varying thickness. Said porous absorbent plates have two different forms, one of them is three-armed star-shaped and the other is circular with alternating recesses and projections spread around the perimeter area, these porous absorbent plates being fitted in the box from its mouth one on top of the other, alternating the two different shapes, so that first of all the three-armed star-shaped plated is placed on the base of the box, a circular plate with peripheral recesses and projections is fitted on this, then another three-armed star plate, and finally another circular plate with recesses and projections on top of this, while the liquid perfume is dispensed on this last plate.

3. The adjustable tabletop air freshener according to claim 1, characterised in that the cap of the box incorporates in its interior plane various vertical ribs, spread equidistantly, that separate the upper absorbent plate from the group of those housed in the box by means of support and pressure, while the actual cap incorporates blade-like ventilation windows so that the air flow passes through the box ventilation grilles, circulating between the absorbent plates and giving off a flow of perfume that issues via the ventilation windows in the cap.

4. The adjustable tabletop air freshener according to claim 1, characterised in that the upper plane of the cover incorporates a self-adhesive label of special plastic material, in the same way as the ventilation slots in the lower portion of the box are provided with a self-adhesive label of the same material covering them up while not in service, said labels incorporating in both cases a tab for pulling off when it is put into use.
